(19) United States
(12) Patent Application Publication
(10) Pub. No.: US 2010/0179372 A1
(43) Pub. Date: Jul. 15, 2010

(54) Method of enhancing skin appearance through the combination of Titan TM and TMR methods

(76) Inventor: Harry A. Glassman, Beverly Hills, CA (US)

Correspondence Address: Cislo & Thomas LLP, 1333 2nd Street, Suite #500, Santa Monica, CA, 90401-4110, US

(21) Appl. No.: 12/319,846

(22) Filed: Jan. 13, 2009

(51) Int. Cl. A61N 5/06; A61B 18/18; A61N 2/00

(52) U.S. Cl. 600/9; 606/9; 607/88

(57) ABSTRACT

A therapy, method, and combination of procedures for tightening facial skin and muscles through the combination of Titan™ and TMR methods. The Titan™ component stimulates collagen resulting in tightened skin. The Titan™ component performs the steps of pre-cooling a surface of an epidermis layer of the skin to prevent burning, applying infrared light to a dermis layer of the skin, wherein the infrared light causes collagen fibers to be heated, and post-cooling the surface of the epidermis layer, the post-cooling contracting the collagen fibers and tightening the skin. The TMR component increases oxygen and nutrients to cells resulting in tightened facial muscles. The TMR component performs the step of applying therapeutic magnetic resonance to the muscles. Through the combination of Titan™ and TMR methods, this non-surgical facelift makes the patient look younger without surgery.

### FIELD OF THE APPLICATION

The present application generally relates to non-invasive skin treatments and more particularly, to a method of enhancing skin appearance through the combination of Titan™ and TMR methods for tightening facial skin and muscles.

### BACKGROUND

Wrinkles often appear as a result of the aging process, but may occur through habitual facial expressions, sun damage, smoking, poor hydration, and various other factors. Smoking, a key factor in the development of wrinkles, robs the facial complexion of oxygen and decreases blood circulation to facial skin resulting in premature lines and wrinkles. Furthermore, smoking consists of repetitive facial movements causing the addition of more wrinkles.

Three forms of treatment have become popular in the fight against wrinkles: tretinoin, botulinum, and restylane. Tretinoin decreases cohesiveness of follicular epithelial cells and stimulates mitotic activity. Botulinum toxin, otherwise known as Botox, decreases muscle activity by blocking the release of acetylcholine at the neuromuscular junction thereby rending the muscle unable to contract. Restylane, a non-animal, stabilized hyaluronic acid, is a wrinkle-resolving gel that is injected into skin to remove wrinkles.

Treatments and products promising to reduce, remove, or prevent age-related wrinkles are big business. Despite great demand, however, most products and treatments have not been proven to give lasting or major positive effects.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with one aspect of the present application, a non-invasive therapy is presented. The therapy includes a Titan™ component for stimulating collagen resulting in tightened skin. The Titan™ component performs the steps of pre-cooling a surface of an epidermis layer of the skin to prevent burning, applying infrared light to a dermis layer of the skin, wherein the infrared light causes collagen fibers to be heated, and post-cooling the surface of the epidermis layer, the post-cooling contracting the collagen fibers and tightening the skin.

In addition, the therapy includes a TMR component for increasing oxygen and nutrients to cells resulting in tightened facial muscles. The TMR component performs the step of applying therapeutic magnetic resonance to the muscles.

In accordance with another aspect of the present application, a method for tightening facial skin and muscles applying a combination of Titan™ and TMR techniques is presented.

In accordance with yet another aspect of the present application, a combination of treatment procedures is presented. The combination of treatment procedures includes a first procedure for tightening facial skin, the first procedure involving pre-cooling the skin, applying heat to the skin, and post-cooling the skin. In addition, the combination of treatment procedures includes a second procedure for tightening facial muscles, the second procedure involving applying therapeutic magnetic resonance to the muscles.

### BRIEF DESCRIPTION OF DRAWINGS

The novel features believed to be characteristic of the application are set forth in the appended claims. In the descriptions that follow, like parts are marked throughout the specification and drawings with the same numerals, respectively. The drawing figures are not necessarily drawn to scale and certain figures may be shown in exaggerated or generalized form in the interest of clarity and conciseness. The application itself, however, as well as a preferred mode of use, further objectives and advantages thereof, will be best understood by reference to the following detailed description of illustrative embodiments when read in conjunction with the accompanying drawings, wherein:

FIG. 1 is an exemplary device capable of providing a Titan™ treatment in accordance with one aspect of the present application;

FIG. 2A depicts illustrative skin having epidermis, dermis, and subcutaneous tissue layers in accordance with one aspect of the present application;

FIG. 2B depicts the exemplary device pre-cooling the skin to protect the epidermis layer in accordance with one aspect of the present application;

FIG. 2C shows the exemplary device heating the dermis layer of the skin to stimulate collagen tightening in accordance with one aspect of the present application;

FIG. 2D illustrates the exemplary device post-cooling the skin to protect the epidermis layer in accordance with one aspect of the present application;

FIG. 3A shows an illustrative collagen fiber strand before Titan™ treatment is applied in accordance with one aspect of the present application;

FIG. 3B shows an illustrative collagen fiber strand after Titan™ treatment is applied in accordance with one aspect of the present application;

FIG. 4 is an exemplary device capable of providing a TMR treatment in accordance with one aspect of the present application;

FIG. 5A depicts an illustrative cell in accordance with one aspect of the present application;

FIG. 5B depicts the illustrative cell treated by the device capable of providing TMR treatment in accordance with one aspect of the present application;

FIG. 5C shows exemplary cells before the TMR treatment in accordance with one aspect of the present application;

FIG. 5D shows exemplary cells after the TMR treatment in accordance with one aspect of the present application;

FIG. 6 illustrates a method combining exemplary Titan™ and TMR treatments in accordance with one aspect of the present application; and

FIG. 7 is an illustrative calendar showing typically treatment cycles for using the method in accordance with one aspect of the present application.

### DETAILED DESCRIPTION

The detailed description set forth below in connection with the appended drawings is intended as a description of presently-preferred embodiments of the application and is not intended to represent the only forms in which the present application may be constructed and/or utilized. The description sets forth the functions and the sequence of steps for constructing and operating the application in connection with the illustrated embodiments. It is to be understood, however, that the same or equivalent functions and sequences may be accomplished by different embodiments that are also intended to be encompassed within the spirit and scope of this application.

Generally described, the present application relates to aging skin. More specifically, the present application relates to a method of enhancing skin appearance through the combination of Titan™ and Therapeutic Magnetic Resonance (TMR) treatments. By tightening the facial skin using a Titan™ treatment and tightening the facial muscles using a TMR treatment, this “non-surgical facelift” makes the patient look younger without surgery. In an illustrative embodiment, a Titan™ component stimulates collagen resulting in tightened skin. The Titan™ component performs the steps of pre-cooling a surface of an epidermis layer of the skin to prevent burning, applying infrared light to a dermis layer of the skin, wherein the infrared light causes collagen fibers to be heated, and post-cooling the surface of the epidermis layer, the post-cooling contracting the collagen fibers and tightening the skin. A TMR component increases oxygen and nutrients to cells resulting in tightened facial muscles. The TMR component performs the step of applying therapeutic magnetic resonance to the muscles.

Areas that have been shown to respond best to the combination of Titan™ and TMR methods are areas of loose, thin skin. Typically, these areas are the patient's neck, face, abdomen, arms, and thighs. Nevertheless, the treatment can be done on a variety of areas.

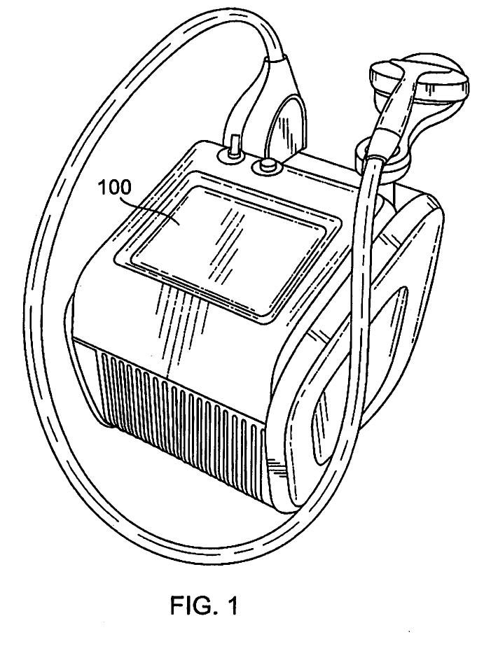

FIG. 1 is an exemplary device 100 capable of providing a Titan™ treatment. Typically, the loss of collagen scaffolding causes skin to become loose, hang, and droop. Titan™ device 100 by Cutera™ tightens the skin on the body without surgery. In addition, Titan™ device 100 has the ability to safely reverse the aging process to provide tighter skin.

On average, the treatment provided for by Titan™ device 100 lasts for about one hour, but can also last up to two and half hours. Little to no pain is associated with the treatment. In addition, no medication for relieving pain is required. Furthermore, there is a minimal risk of infection and no scarring. There is no down time associated with this treatment.

Repeated uses of Titan™ device 100 may be required. While improvements may be seen after a single treatment, most people typically require a series of two treatments to reach their goal with the results being permanent.

Titan™ device 100 generally utilizes infrared light technology to safely heat the water found in the mid to deep dermis to cause collagen contraction as will be provided in more detail below. This process also activates other skin components to make new collagen and help restore the skin's collagen scaffolding causing the skin to tighten naturally.

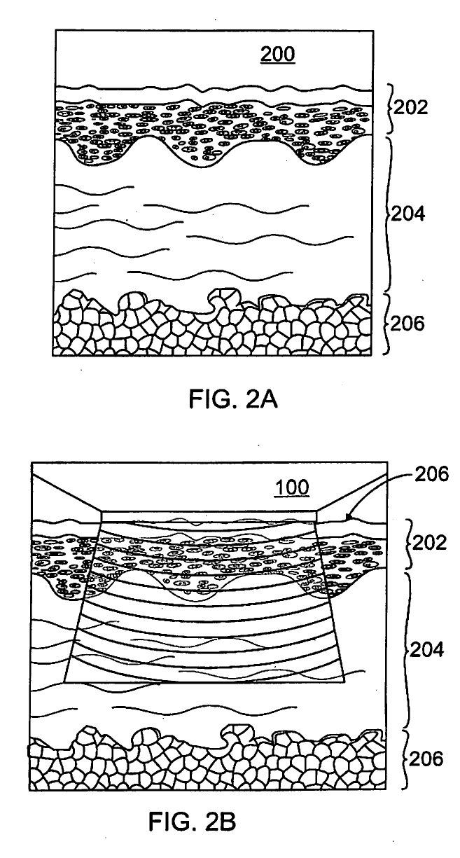

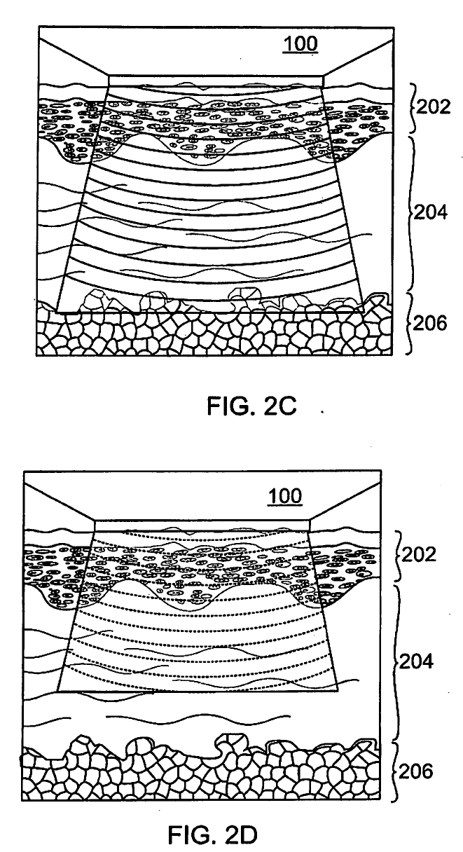

FIG. 2A depicts illustrative skin 200 having epidermis 202, dermis 204, and subcutaneous tissue 206 layers. Epidermis 202 is the outermost layer of the skin 200, composed of terminally differentiated stratified squamous epithelium acting as the body's major barrier against an inhospitable environment. Dermis 204 is a layer of skin 200 between the epidermis 202 and subcutaneous tissues 204 and is composed of two layers, the papillary and reticular dermis. Structural components of the dermis 204 are collagen, elastic fibers, and extrafibrillar matrix. Subcutaneous tissue 206 is a layer of fat that lies between the dermis 204 of the skin 200 and underlying fascia. Subcutaneous fat insulates the body, absorbs trauma, and is a reserve energy source. This tissue 206 may be further divided into two components, the actual fatty layer, or panniculus adiposus, and a deeper vestigial layer of muscle, the panniculus carnosus.

Titan™ device 100, as depicted in FIG. 2B, provides Titan™ treatment by initially pre-cooling skin 200. More specifically, the pre-cooling is applied to the epidermis layer 202 to prevent burning of the skin 200. Titan™ device 100 can use fans or some other type of cooling element to pre-cool skin 200.

By cooling the epidermis layer 202, varying degrees of thermal injury can be achieved at varying depths in the dermis layer 204 while preserving the viability of the epidermis layer 202. Typically, cooling times may vary. Preferably, however the pre-cooling time lasts for about five seconds.

FIG. 2C shows exemplary Titan™ device 100 heating dermis layer 204 of skin 200 to stimulate collagen tightening. Preferably, a specially filtered infrared light is used to heat dermis layer 204 in a safe, uniform fashion. The infrared light can penetrate into the dermis layer 204 to about two point five millimeters to stimulate collage tightening. Typically, as this heat is being applied to the deep dermis layer 204, epidermis layer 202 can be simultaneously protected by constant contact with Titan™ device 100, which cools the skin 200 from the outside. Throughout the course of this procedure, the heat from the infrared energy will begin stimulating the growth of new collagen in the dermis layer 204, while also tightening the existing collagen in the body. This increase in collagen levels is essential to creating better skin elasticity and volume. Thus over time, Titan™ skin 200 tightening treatment will begin to reveal natural, noticeable results.

Alternatively, Titan™ device 100 can also use other elements for applying heat to the dermis layer 204 of skin 200. As such, lasers or some other electromagnetic radiation source may also be used instead of the filtered infrared light. Furthermore, Titan™ device 100 may forego simultaneously cooling skin 200 while applying heat.

After heating dermis layer 204 to stimulate the growth of new collagen, Titan™ device 100 applies post-cooling to skin 200. By post-cooling epidermis layer 202, skin 200 is protected.

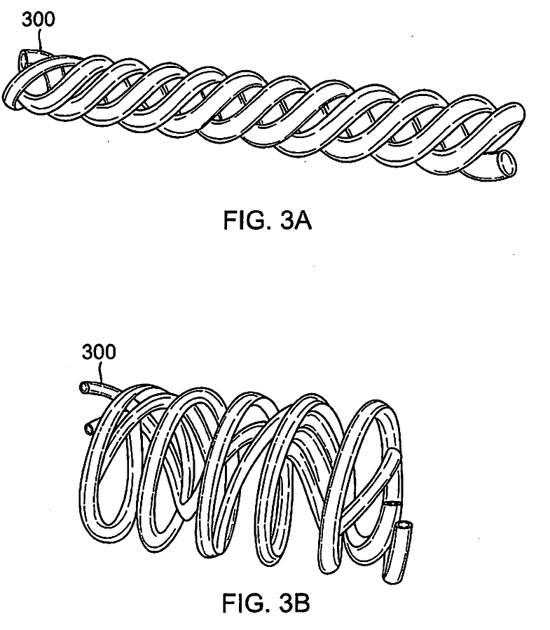

Within dermis layer 204, FIG. 3A shows an illustrative collagen fiber strand 300 before Titan™ treatment is applied in accordance with one aspect of the present application. Applying heat through the infrared light cause the collagen fiber strand 300 to contract as depicted in FIG. 3B. The overall structure of the dermis layer 204 contracts due to collagen denaturing and consequently, skin 200 is tightened. Through Titan™ device 100, immediate reduction of swelling, significant reduction in down time from injuries, and even dramatic success for fibromyalgia and phantom limb pain is provided.

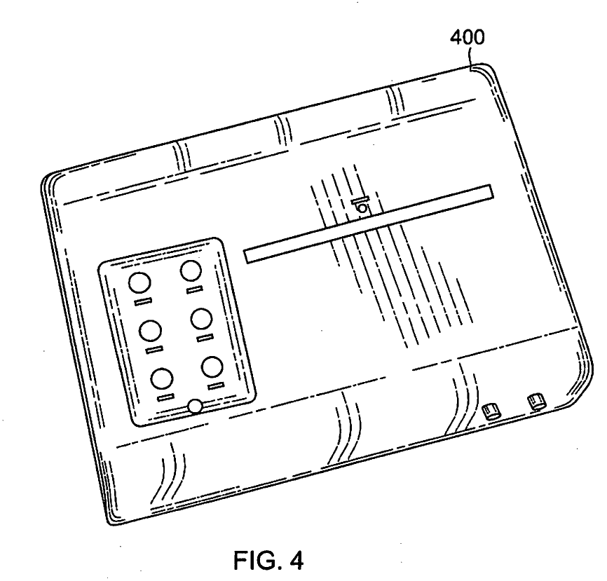

By combining treatments from exemplary Titan™ device 100 with exemplary TMR device 400, as shown in FIG. 4, both facial skin and facial muscles may be tightened enhancing skin appearance through non-invasive treatments. TMR device 400 is cleared by the United States Food and Drug Administration (FDA) to reduce pain when applied topically. Benefits of TMR device 400 include reduction in swelling, bruising, and improvements in wound healing.

Using TMR device 400 requires no surgery, drugs, needles, narcotics with side effects, withdrawal symptoms, or overdose risks. Typically, TMR device 400 provides a gentle tingling sensation. TMR device 400, when applied to skin 200 overlying the facial muscles, causes sustained muscular contraction that improves facial muscle tone and tightens the facial muscles consequently making the individual look younger.

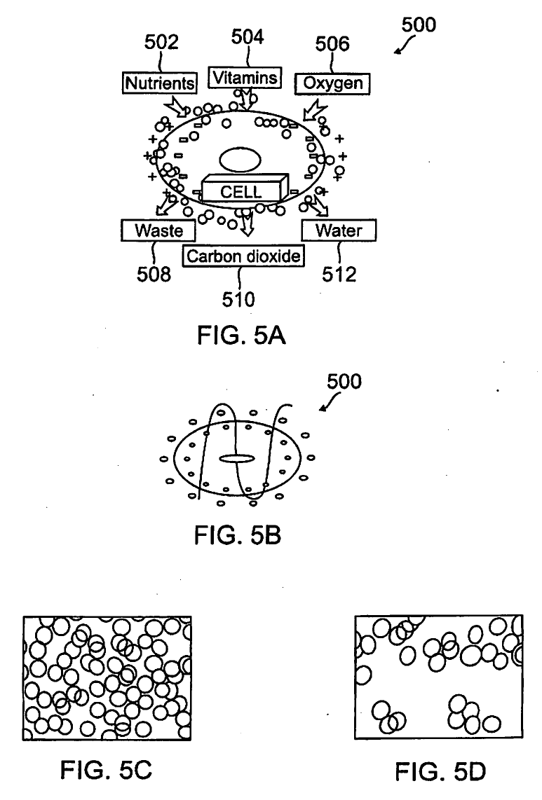

FIG. 5A depicts an illustrative cell 500 in accordance with one aspect of the present application. Many cells 500 make up muscles which are surrounded by skin 200. Cell 500 includes a membrane that envelops cell 500, separates its interior from its environment, regulates what moves in and out, and maintains the electric potential of cell 500. As depicted, cell 500 may take in nutrients 502, vitamins 504, and oxygen 506, while expelling waste 508, carbon dioxide 510, and water 512.

Important to TMR device 400 are the mitochondria which are located within cell 500. Mitochondria are self-replicating organelles that occur in various numbers, shapes, and sizes in the cytoplasm of cell 500. Mitochondria play a critical role in generating energy in cell 500. Mitochondria generate energy by the process of oxidative phosphorylation which consists of utilizing oxygen to release energy stored in cellular nutrients to generate ATP.

When applied, TMR device 400 provides therapeutic magnetic resonance to cell 500 as depicted in FIG. 5B. Typically, pulsating signals are applied. These signals may use a “saw-tooth profile” and resemble small frequencies associated with the magnetic earth field. Bundles of frequency having resonance effect may also be used.

Under the influence of these magnetic fields produced by TMR device 400, red blood cells deliver more oxygen to every single cell 500. Magnetic fields are an important requirement for improved cell 500 function, particularly in the case of cells 500 in a state of “fatigued function” as shown in FIG. 5C. Such cells 500 are fatigued due to lack of oxygen and nutrients and no longer function properly and are in danger of cell 500 death.

Through the therapeutic application of pulsating magnetic fields, these cells 500 increasingly absorb fuel again. The mitochondria will begin to produce more energy and the cell membrane pump will increase its activity. In the process, the membrane potential is increased, together with cell tension so that the cell gains its activity and can resume its tasks as shown in FIG. 5D. These processes are due primarily to the increased oxygen supply resulting from improvement of the membrane potential.

In addition, pulsating magnetic fields generated by TMR device 400 produce tiny electromagnetic impulses in the body. These electromagnetic impulses in turn produce surges at the transmission points of nerve and muscle cells. By weakening nerve impulses to the muscle cells, they lose their tension and are able to relax. This has a positive effect on the entire organism including muscle relaxation and improved blood circulation. All muscle tension and even spasm can be relieved or even eliminated.

The increased circulation, together with the above mentioned improved oxygen supply and improved metabolism, produces an ideal combination. Relaxation of muscles combined with improved circulation can often eliminate the cause of the pain rather than just masking the symptom.

Optimal oxygen supply facilitates a properly functioning immune system. Moreover, appropriate pulsating magnetic fields boost the amount of calcium available to the immune system. Since bodies consist mainly of water, there are sufficient low-charged hydrogen ions present in the tissue. These are the smallest ions present in the body and under the influence of magnetic fields; they begin to “migrate”. Two of these hydrogen ions replace one calcium ion, bound in the tissue allowing calcium to be released and available to the immune system to do its important job.

While both Titan™ device 100 and TMR device 400 can be used as separate devices, both treatments may also be combined into a single device. This device would allow for Titan™ treatments as well as TMR treatments. By combining treatments from exemplary Titan™ device 100 with exemplary TMR device 400, both facial skin and facial muscles may be tightened enhancing skin appearance through non-invasive treatments.

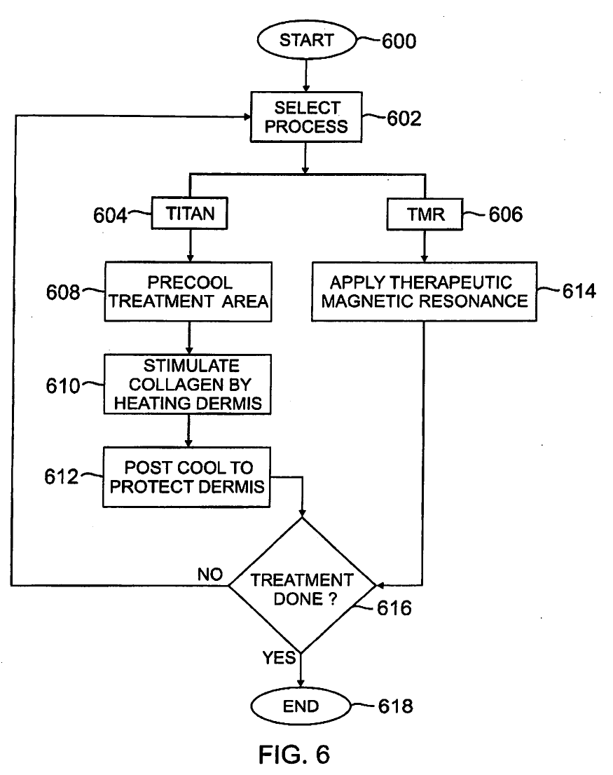

With reference now to FIG. 6, a method for combing an exemplary Titan™ treatment 604 and an exemplary TMR treatment 606 is presented in accordance with one aspect of the present application. One skilled in the relevant art will appreciate that there are numerous ways to combine both treatments and this method does not limit the scope of this application. Furthermore, the method presented herein can include additional, fewer, or similar processes.

Beginning at block 600, the method of applying the Titan™ treatment, TMR treatment, or both is provided for. At block 602, a user can make a selection of which process to use. The user can select one treatment or both treatments. When the Titan™ treatment is selected at block 602, the Titan™ process is applied to skin 200 at block 604. At block 608, the treatment area is pre-cooled. Pre-cooling the epidermis layer 202 of skin 200 prevents burning of skin 200. At block 610, the collagen within dermis layer 204 is stimulated through heat. Preferably, an infrared light is applied. The Titan™ process may apply cooling techniques simultaneous to the application of heat. In turn, the Titan™ treatment provides post-cooling which contracts the collagen fibers 300 and tightens skin 200.

When the TMR treatment is selected at block 602, the TMR process is applied to skin 200 at block 606. The TMR process at block 614 increases oxygen and nutrients to cells 500 resulting in tightened facial muscles. The TMR process performs the step of applying therapeutic magnetic resonance to the muscles.

Both the Titan™ 604 and TMR 606 treatments thereafter go to decision block 616. At decision block 616, the user may continue the method if the treatment is not done by returning to block 602. When the treatment has been completed, the user can end the method at block 618.

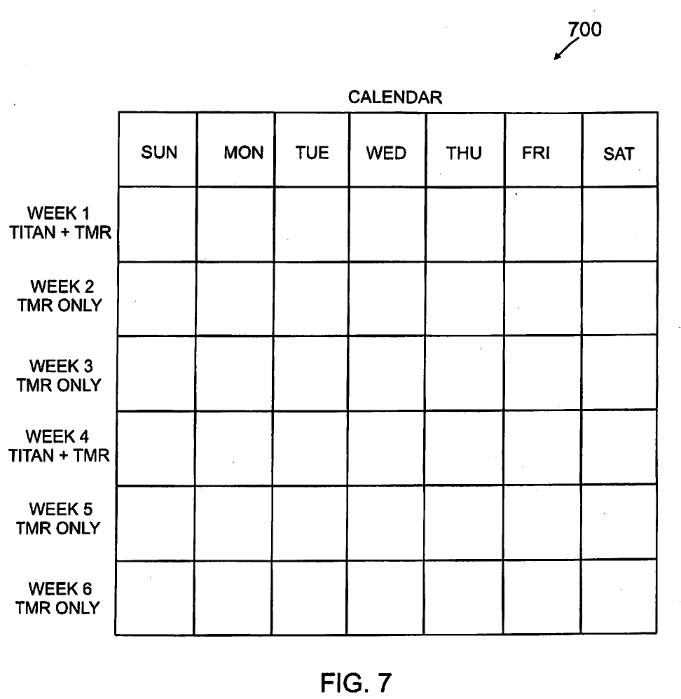

FIG. 7 is an illustrative calendar 700 showing typically treatment cycles for using the method in accordance with one aspect of the present application. The user may apply both Titan™ process 604 along with TMR process 606 for the first week. Thereafter, the user may apply the TMR process 606 for the next two weeks. On the fourth week, the user again may apply both Titan™ process 604 along with TMR process 606 followed by two additional weeks of TMR process 606. In total, the procedure can last for six weeks as indicated by illustrative calendar 700.

Alternatively, the user can repeatedly apply both Titan™ 604 and TMR 606 processes for one week followed by TMR processes 606 for two weeks thereafter until both facial skin 200 and muscles have been tightened. Preferably, Titan™ process 604 is applied before TMR process 606.

In accordance with one aspect of the present application, a non-invasive therapy is presented. The therapy includes a Titan™ component for stimulating collagen resulting in tightened skin. The Titan™ component performs the steps of pre-cooling a surface of an epidermis layer of the skin to prevent burning, applying infrared light to a dermis layer of the skin, wherein the infrared light causes collagen fibers to be heated, and post-cooling the surface of the epidermis layer, the post-cooling contracting the collagen fibers and tightening the skin.

In addition, the therapy includes a TMR component for increasing oxygen and nutrients to cells resulting in tightened facial muscles. The TMR component performs the step of applying therapeutic magnetic resonance to the muscles.

In accordance with another aspect of the present application, a method for tightening facial skin and muscles applying a combination of Titan™ and TMR techniques is presented.

In accordance with yet another aspect of the present application, a combination of treatment procedures is presented. The combination of treatment procedures includes a first procedure for tightening facial skin, the first procedure involving pre-cooling the skin, applying heat to the skin, and post-cooling the skin. In addition, the combination of treatment procedures includes a second procedure for tightening facial muscles, the second procedure involving applying therapeutic magnetic resonance to the muscles.

The foregoing description is provided to enable any person skilled in the relevant art to practice the various embodiments described herein. Various modifications to these embodiments will be readily apparent to those skilled in the relevant art, and generic principles defined herein may be applied to other embodiments. Thus, the claims are not intended to be limited to the embodiments shown and described herein, but are to be accorded the full scope consistent with the language of the claims, wherein reference to an element in the singular is not intended to mean “one and only one” unless specifically stated, but rather “one or more.” All structural and functional equivalents to the elements of the various embodiments described throughout this disclosure that are known or later come to be known to those of ordinary skill in the art are expressly incorporated herein by reference and intended to be encompassed by the claims. Moreover, nothing disclosed herein is intended to be dedicated to the public regardless of whether such disclosure is explicitly recited in the claims.

What is claimed is:

1. A non-invasive therapy comprising:
a Titan™ component for stimulating collagen resulting in tightened skin, the Titan™ component performing the steps of:
pre-cooling a surface of an epidermis layer of the skin to prevent burning;
applying infrared light to a dermis layer of the skin, wherein the infrared light causes collagen fibers to be heated;
post-cooling the surface of the epidermis layer, the post-cooling contracting the collagen fibers and tightening the skin; and
a TMR component for increasing oxygen and nutrients to cells resulting in tightened facial muscles, the TMR component performing the step of:
applying therapeutic magnetic resonance to the muscles.

2. The non-invasive treatment therapy of claim 1, wherein the Titan™ component is applied before the TMR component.

3. A method for tightening facial skin and muscles applying a combination of Titan™ and TMR techniques.

4. The method for tightening facial skin and muscles of claim 3 comprising initially applying both Titan™ and TMR techniques.

5. The method for tightening facial skin and muscles of claim 3 comprising:
repeatedly:
applying both Titan™ and TMR techniques for one week; and
applying TMR techniques for two weeks thereafter;
until both facial skin and muscles have been tightened.

6. The method for tightening facial skin and muscles of claim 3 comprises applying both Titan™ and TMR techniques for six weeks.

7. The method for tightening facial skin and muscles of claim 3, wherein the Titan™ technique comprises:
cooling a surface of the skin to prevent burning; and
simultaneously applying infrared light to the skin, wherein the infrared light causes collagen fibers to be heated.

8. The method for tightening facial skin and muscles of claim 3, wherein the TMR technique comprises applying therapeutic magnetic resonance to the muscles.

9. The method for tightening facial skin and muscles of claim 8, wherein applying therapeutic magnetic resonance to the muscles causes reduction in swelling, reduction in bruising, and improved wound healing.

10. The method for tightening facial skin and muscles of claim 8, wherein applying therapeutic magnetic resonance to the muscles causes stimulation in cells within the muscles.

11. A combination of treatment procedures comprising:
a first procedure for tightening facial skin, the first procedure involving pre-cooling the skin, applying heat to the skin, and post-cooling the skin; and
a second procedure for tightening facial muscles, the second procedure involving applying therapeutic magnetic resonance to the muscles.

12. The combination of treatment procedures of claim 11, wherein applying heat to the skin comprises using an infrared light.

13. The combination of treatment procedures of claim 11, wherein applying heat to the skin stimulates collagen tightening.

14. The combination of treatment procedures of claim 13, wherein applying heat to the skin stimulating collagen tightening is a result of contraction of fiber within the collagen.

15. The combination of treatment procedures of claim 11, wherein applying heat to the skin raises the temperature of a dermis layer of the skin.

16. The combination of treatment procedures of claim 11, wherein the first procedure for tightening facial skin is a Titan™ procedure.

17. The combination of treatment procedures of claim 11, wherein applying therapeutic magnetic resonance to the muscles comprises pulsating signals.

18. The combination of treatment procedures of claim 11, wherein applying therapeutic magnetic resonance to the muscles comprises saw-tooth impulses.

19. The combination of treatment procedures of claim 11, wherein applying therapeutic magnetic resonance to the muscles increases oxygen and nutrients to cells within the muscles.

20. The combination of treatment procedures of claim 11, wherein the second procedure for tightening facial muscles is a TMR procedure.
